(19) United States
(12) Patent Application Publication
(10) Pub. No.: US 2008/0286375 A1
(43) Pub. Date: Nov. 20, 2008

(54) Method for Preparing Sustained-Release Microparticles Comprising Sucrose Acetate Isobutyrate

(75) Inventors: Eun Seong Lee, Gyeonggi-do (KR); Hyeok Lee, Gyeonggi-do (KR); Jung Ju Kim, Gyeonggi-do (KR)

Correspondence Address: SUGHRUE MION, PLLC, 2100 PENNSYLVANIA AVENUE, N.W., SUITE 800, WASHINGTON, DC, 20037, US

(73) Assignee: Amorepacific Corporation, Seoul (KR)

(21) Appl. No.: 12/093,872

(22) PCT Filed: Nov. 15, 2002
(86) PCT No.: PCT/KR2006/004799

§ 371 Date: May 15, 2008

(30) Foreign Application Priority Data
Nov. 15, 2005 (KR) 10-2005-0108884

(51) Int. Cl. A61K 9/14; A61K 38/16; A61K 38/47; A61K 38/28; A61K 38/14; A61K 38/21; A61K 39/395; A61K 38/20; A61K 38/48; A61K 38/44; A61K 39/12; A61K 38/30; A61K 38/27; A61K 38/18; A61P 43/00

(52) U.S. Cl. 424/499; 514/12; 424/94.61; 514/3; 514/8; 424/85.4; 424/145.1; 424/85.2; 424/94.64; 424/94.4; 424/204.1; 424/142.1; 424/133.1

(57) ABSTRACT

A sustained release microparticles which is capable of releasing a protein drug continuously over a long period of time without initial burst release of the drug can be simply prepared by a method including the steps of a) dissolving a protein drug in an aqueous solution to obtain a water phase; b) dissolving sucrose acetate isobutyrate (SAIB) and a biodegradable polymer in an organic solvent to obtain an oil phase; c) adding the water phase obtained in step a) to the oil phase obtained in step b) to form a primary emulsion; and d) adding the primary emulsion to an external aqueous continuous phase to form a secondary emulsion and recovering the solid product formed in the secondary emulsion.

### TECHNICAL FIELD

The present invention relates to a method for preparing sustained-release microparticles comprising sucrose acetate isobutyrate.

### BACKGROUND ART

There have been reported numerous studies to develop sustained-release microparticles of a protein drug that exhibit a desirable release pattern of the drug without initial burst release.

For example, Korean Patent No. 321,854 discloses a method for controlling the drug release rate by mixing sustained-release microparticles of a protein drug prepared using an easily biodegradable polymer containing carboxylic terminal group and microparticles containing the same drug using a biodegradable polymer containing dodecyl terminal group that biodegrade much slower; Korean Patent No. 392,501 discloses a method for preparing sustained-release microparticles using two or more biodegradable polymers; Korean Publication Patent No. 2005-1896 discloses a method for preparing microparticles having varying compositions by continuously spraying and drying a fluid containing more than two different biodegradable polymers.

However, the above techniques are cumbersome in that the fluid and oil phases must be prepared separately and the loading efficiency of the drug is not satisfactory, besides the problem that in the case for a macromolecular protein drug, the total release amount of the protein drug becomes dependent on the molecular weight of the biodegradable polymer (See, Y. Yeo, Arch. Pharm. Res. 27, 1-12, 2004; and V. R. Sinha, J. Control. Release, 90, 261-280, 2003).

U.S. Pat. No. 4,652,441 discloses a technique for suppressing the initial burst release of the drug and enhancing the loading efficiency of aqueous peptide-containing microparticles by using a viscous gelatin. However, the viscosity of the gelatin obtained by treating animal collagen with an acid or base changes unpredictably depending on the temperature.

U.S. Pat. No. 6,120,787 discloses a method of preparing polymer microparticles capable of achieving continuous drug release for one month by coating a protein drug with starch, and then coating the starch-coated drug with a biodegradable polymer. However, this method is cumbersome in that two different coating steps are required and the starch-coated protein drug particles tend to agglomerate before the second coating step.

### DISCLOSURE OF INVENTION

### Technical Problem

Accordingly, an object of the present invention is to provide a method for preparing sustained-release microparticles comprising sucrose acetate isobutyrate, which is capable of releasing the drug continuously over a long period of time without initial burst release of the drug.

### Technical Solution

In accordance with one aspect of the present invention, there is provided a method for preparing a sustained-release microparticle comprising:

a) dissolving a protein drug in an aqueous solution to obtain a water phase;

b) dissolving sucrose acetate isobutyrate (SAIB) and a biodegradable polymer in an organic solvent to obtain an oil phase;

c) adding the water phase obtained in step a) to the oil phase obtained in step b) to form a primary emulsion; and

d) adding the primary emulsion to an external aqueous continuous phase to form a secondary emulsion and recovering the solid product formed in the secondary emulsion.

### BRIEF DESCRIPTION OF THE DRAWINGS

The above and other objects and features of the present invention will become apparent from the following description of the invention, when taken in conjunction with the accompanying drawing which shows:

FIG. 1: a scanning electron microscopic image of the microparticles prepared in Example 1 of the present invention.

FIG. 2: a graph showing the drug release rates by the microparticles prepared in Example 1 and Comparative Example 1 of the present invention versus the time.

FIG. 3: a graph showing the drug release rates of the microparticles prepared in Examples 4 to 8 of the present invention.

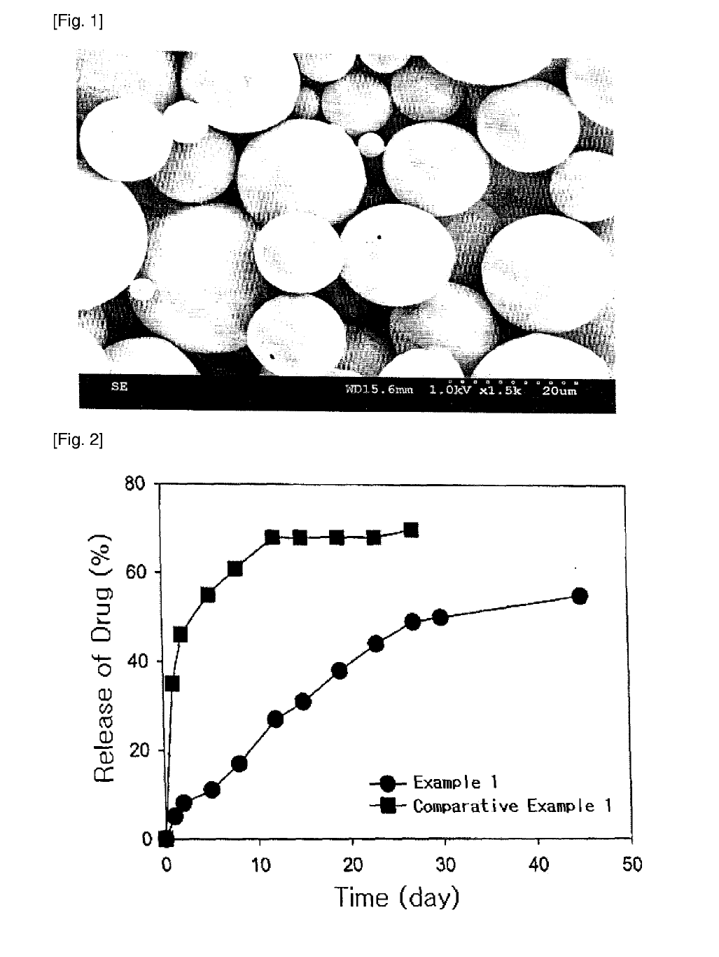

### MODE FOR THE INVENTION

The method of preparing microparticles according to the present invention is described in detail as follows:

Step 1: Preparation of a Water Phase Containing a Protein Drug.

The title water phase is prepared by dissolving a protein drug in a solvent such as distilled water, phosphate buffer solution, borate buffer solution and Tris-HCl buffer solution. In this step, additives selected from the group consisting of a release-controlling agent, a stabilizer and a mixture thereof are added to the aqueous solution as needed.

Representative examples of the release-controlling agents used in the present invention include hydrophilic agent such as polyethylene glycol, polyoxyethylene sorbitan fatty acid ester, glyceryl monooleate, sorbitan fatty acid ester, hyaluronic acid, chondroichin sulfate, polyvinylalcohol, starch, bovine serum albumin, chitosan, alginic add, pectin, curdlan, gelatin, dextran, levan, glucan, polyhistidine, polylysine, poloxamer, glyceryl palmitostearate, benzylbenzoate, ethyloleate and the like; lipophilic agent such as soybean oil, cotton seed oil, sesame oil, peanut oil, canola oil, corn oil, coconut oil, rapeseed oil, theobroma oil and the like; glycerin; mannitol; and a mixture thereof. Preferably, the release-controlling agent can be selected from the group consisting of polyethylene glycol, poloxamer, polyoxyethylene sorbitan fatty add ester, glyceryl monooleate, sorbitan fatty acid ester, hyaluronic acid, chondroichin sulfate, chitosan, alginic acid, pectin, gelatin, dextran, bovine serum albumin, sesame oil, glycerin and mannitol; more preferably, polyethylene glycol.

In accordance with the present invention, the weight ratio of protein drug:release-controlling agent ranges from 1:0.01 to 1:10, preferably, from 1:0.2 to 1:5. When a polyethylene glycol is used as the release-controlling agent, it preferably has a weight-average molecular weight of 1,000 to 20,000.

The drug stabilizer that may be used in the present invention is selected from the group consisting of a viscous water-soluble polymer, a cyclodextrin derivative and a mixture thereof.

The viscous water-soluble polymer must be highly biocompatible, and representative examples of thereof include starch, cellulose, hemicellulose, pectin, lignin, chitosan, xanthan gum, alginic acid, pullulan, curdlan, gelatin, dextran, levan, hyaluronic acid, glucan, collagen, salt thereof and a mixture thereof. Preferred is soluble starch, potato starch, hyaluronic acid or gelatin, more preferably, soluble starch or hyalurone acid. The viscous water-soluble polymer is added to the aqueous solution to a concentration ranging from 0.1 to 10% (w/v).

The viscous water-soluble polymer serves to form a viscous film protecting the protein drug from degeneration in the boundary region between the organic solvent and water phase, and it further contributes to the delay of the drug release.

Representative examples of cyclodextrin derivatives include 3-mono-o-methyl-cyclodextrin, 2,6-di-o-methyl-cyclodextrin, 2,3,6-tri-o-methyl-cyclodextrin, 2-hydroxyethyl-cyclodextrin, 2-hydroxypropyl-cyclodextrin, 3-hydroxypropyl-cyclodextrin, 6-o-glucosyl-cyclodextrin, 6-o-maltosyl-cyclodextrin, 6-o-dimaltosyl-cyclodextrin, 2,6-di-o-ethyl-cyclodextrin, 2,3,6-tri-o-ethyl-cyclodextrin, 2,3-di-o-hexanoyl-cyclodextrin, 2,3,6-tri-o-acetyl-cyclodextrin, 2,3,6-tri-o-propanoyl-cyclodextrin, 2,3,6-tri-o-butanoyl-cyclodextrin, 2,3,6-tri-o-hexanoyl-cyclodextrin, 6-o-carboxymethyl-cyclodextrin, sulfated cyclodextrin, sulfobutyl-cyclodextrin, derivatives thereof and a mixture thereof, preferably 2-hydroxypropyl-cyclodextrin, 2,6-di-o-methyl-cyclodextrin, 6-o-maltosyl-cyclodextrin, and β-cyclodextrin sulfobutyl ether sodium (CAPTISOL™) as a derivate of sulfobutyl-cyclodextrin, preferably β-cyclodextrin sulfobutyl ether sodium.

The weight ratio of protein drug:cyclodextrin derivative ranges from 1:0.1 to 1:20.

The cyclodextrin derivative serves as a protein drug stabilizer and protects the drug from degeneration by incorporating the protein drug in its cavity to form a host-guest component.

The protein drug used in the present invention consists of two or more amino acids and preferably has a weight-average molecular weight of 200 to 100,000 daltons.

Representative examples of protein drugs that can be used in the prevent invention include lysozyme, human growth hormone, insulin, bovine growth hormone, porcine growth hormone, growth hormone releasing peptide, B-cell factor, T-cell factor, granulocyte-colony stimulating factor, granulocyte macrophage-colony stimulating factor (GM-CSF), macrophage-colony stimulating factor (M-CSF), erythropoietin, bone morphogenic protein, interferon, atriopeptin-III, monoclonal antibody, macrophage activating factor, interleukin, tumor degenerating factor, insulin-like growth factor, epidermal growth factor, tissue plasminogen activator, urokinase, protein A allergy inhibiting factor, cell necrosis glycoprotein, immunotoxin, lympotoxin, tumor necrosis factor, tumor inhibitory factor, transforming growth factor, alpha-1 antitrypsin, albumin and its fragment polypeptide, apolipoprotein-E, factor VII factor VIII factor IX, pancreatic polypeptide, protein C, C-reactive protein, renin inhibitor, collagenase inhibitor, superoxide dismutase, platelet derived growth factor, osteogenic growth factor, osteogenesis stimulating protein, calcitonin, atriopeptin, cartilage including factor, connective tissue activating factor, follicle-stimulating hormone, luteinizing hormone, luteinizing hormone releasing hormone, neurotrophic factor, parathyroid hormone, secretin, somatomedin, adrenocorticotropic hormone, glucagon, cholecystokinin, gastrin-releasing peptide, corticotropin releasing factor, thyroid stimulating hormone, various virus, bacteria, monoclonal or polyclonal antibodies against toxin, and various virus derived vaccine antigen; human leutenizing hormone releasing hormone homology such as leuprorelin acetate, goserelin acetate, nafarelein acetate, buserelin acetate, gonadorelin; Humira(adalimumab); Remicade(infliximab); octreotide acetate; a salt thereof and a mixture thereof.

Step 2: Preparation of an Oil Phase

An oil phase is prepared by dissolving sucrose acetate isobutyrate (SAIB) and a biodegradable polymer in an organic solvent. The weight ratio of biodegradable polymer:SAIB ranges from 1:0.1 to 1:5 preferably from 1:0.1 to 1:2.

Since SAIB alone is not capable of forming microparticles in the primary emulsion, it is mixed with a biodegradable polymer. Also, when SAIB is used as a sustained-release gel for injection, an organic solvent such as ethanol is added to SAIB to control its viscosity. The microparticles containing SAIB according to the present invention, however, does not necessarily contain an organic solvent.

Representative examples of biodegradable polymer used in the present invention include poly(acryloyl hydroxyethyl) starch, polybutylene terephthalate-polyethylene glycol copolymer, chitosan and derivatives thereof, polyorthoester-polyethylene glycol copolymer, polyethylene glycol terephthalate-polybutylene terephthalate copolymer, poly sebacic anhydride, pullulan and derivatives thereof, starch and derivatives thereof, cellulose acetate and derivatives thereof, polyanhydride, polylactic acid, polyglycolic acid, polylactic acid-polyglycolic acid copolymer, polycaprolactone, polycarbonate, polybutadiene, polyesters, polyhydroxybutyric acid, polymethyl methacrylate, polymethacrylic acid ester, polyorthoester, polyvinyl acetate, polyvinyl alcohol, polyvinyl butyral, polyvinyl formal, hyaluronic acid, lecithin, starch, protein (albumin, casein, collagen, fibrin, fibrinogen, gelain, hemoglobin, transferrin, jein) and a mixture thereof; preferably hyaluronic acid, lecithin, starch, polylactic acid, polyglycolic acid, polylactic acid-polyglycolic acid copolymer and polycaprolactone; more preferably polylactic acid-polyglycolic acid copolymer.

The above polymers are known have excellent biocompatibility and biodegradability and they are metabolized into water and carbon dioxide in vivo via the citric acid cycle (see, S. J. Holland et al., J. Controlled Release, 4, 155-180, 1986).

The biodegradable polymer preferably has a weigh-average molecular weight ranging from 2,000 to 100,000 daltons. The biodegradable polymer is dissolved in an organic solvent to a concentration in the range of 5 to 60% (w/v).

The organic solvent should be miscible without phase-separation with the biodegradable polymer and SAIB, and it may be dichloromethane, ethylacetate, dimethylsulfoxide, dimethylformamide, chloroform, alcohol, acetone or a mixture thereof.

Step 3: Preparation of Primary Emulsion

An emulsion is prepared by adding the water phase prepared in step (1) to oil phase prepared in step (2). The volume ratio of the water phase:oil phase ranges from 1:3 to 1:30, preferably from 1:3 to 1:15.

Step 4: Preparation of Microparticles

Microparticles are prepared by adding the prima emulsion prepared in step (3) to an external aqueous continuous phase to form a secondary emulsion, and separating solid formed in the secondary emulsion by a conventional method such as filtration and centrifugal purification. The volume ratio of the primary emulsion:external aqueous continuous phase ranges from 1:50 to 1:500, preferably from 1:100 to 1:300.

The external aqueous continuous phase used in the present invention may be an aqueous solution of polyvinyl alcohol, methyl cellulose, cetyltrimethyl ammonium bromide, sodium dodecyl sulphate or polyoxyethylene sorbitan monooleate, and preferred is an aqueous polyvinyl alcohol solution. The solute concentration of the aqueous solution used in the external aqueous continuous phase ranges from 0.1 to 5% (w/v), and preferably, 0.3 to 2% (w/v).

When the external aqueous continuous phase is an aqueous polyvinyl alcohol solution, the polyvinyl alcohol has a weight-average molecular weight of 10,000 to 100,000 daltons, preferably 13,000 to 50,000 daltons, and a degree of hydrolysis of 75 to 95%, preferably 83 to 89%.

In order to control the osmotic pressure of the microparticles, sodium hydrochloride can be added to the aqueous polyvinyl alcohol solution. In this case, the Concentration of sodium hydrochloride is preferably 0.1 to 10% (w/v) based on the aqueous polyvinyl alcohol solution.

The microparticles prepared as described above have a mean particle diameter of 0.1 to 200 μm, preferably 10 to 100 μm. The microparticles may comprise a drug in an amount of 1 to 40 wt % based on the total weight of the microparticles.

The following Examples are given for the purpose of illustration only, and are not intended to limit the scope of the invention.

### EXAMPLE 1

A water phase was prepared by dissolving 100 mg of lysozome in 0.5 ml of phosphate buffer (pH 5.1), while an oil phase was prepared by dissolving 300 mg of RG 502H (Boehringer Ingelheim Inc) (polylactic acid-polyglycolic acid copolymer (molar ratio of lactic acid:glycolic acid=50:50)) and 100 mg of SAIB in 3 ml of dichloromethane. The water and oil phase thus prepared were combined (volume ratio of the water phase:the oil phase=1:6) and stirred vigorously to obtain a primary emulsion. An external aqueous continuous phase containing 0.5% (w/v) polyvinyl alcohol and 0.9% (w/v) sodium hydrochloride was placed in a homogenizer operating at 4,000 rpm, the above primary emulsion was slowly added thereto until the volume ratio of the primary emulsion to the external aqueous continuous phase reached 1:200, and the resulting mixture was homogenized for 5 min to obtain a second emulsion. The resulting emulsion was then centrifuged for 2 min at 3000 rpm to separate a solid product which was dried in a freeze dryer for 48 hours to obtain microparticles.

### EXAMPLE 2

Microparticles were prepared according to the same method as in Example 1 except for preparing the oil phase by dissolving 266 mg of RG 502H and 133 mg of SAIB in 3 mg of dichloromethane.

### EXAMPLE 3

Microparticles were prepared according to the same method as in Example 1 except for preparing the oil phase by dissolving 200 mg of RG 502H and 200 mg of SAIB in 3 mg of dichloromethane.

### EXAMPLE 4

Microparticles were prepared according to the same method as in Example 1 except for preparing the water phase by further dissolving starch (2.5% (w/v)).

### EXAMPLE 5

Microparticles were prepared according to the same method as in Example 1 except for preparing the water phase by further dissolving hyaluronic acid (0.5% (w/v)).

### EXAMPLE 6

Microparticles were prepared according to the same method as in Example 1 except for preparing the water phase by further dissolving polyethylene glycol (weigh-average molecular weight: 2000) (5.0% (w/v)) and CAPTISOL™ (CyDex Inc.) (10% (w/v)).

### EXAMPLE 7

Microparticles were prepared according to the same method as in Example 1 except for preparing the water phase by further dissolving aqueous starch (2.5% (w/v)), polyethylene glycol (weigh-average molecular weight: 2000) (5.0% (w/v)) and CAPTISOL™ (10% (w/v)).

### EXAMPLE 8

Microparticles were prepared according to the same method as in Example 1 except for preparing the water phase by further dissolving hyaluronic acid (0.5% (w/v)), polyethylene glycol (weigh-average molecular weight: 2000) (5.0% (w/v)) and CAPTISOL™ (10% (w/v)).

### COMPARATIVE EXAMPLE 1

Microparticles were prepared according to the same method as in Example 1 except for dissolving only 400 mg of RG 502H in 3 ml of dichloromethane.

### TEST EXAMPLE 1

### Evaluation of Drug Loading Efficiency of Microparticles

10 mg of each of the microparticles prepared in Examples 1 to 8 and Comparative Example was weighed accurately in a test tube with a cap and a completely dissolved in 0.5 mg of dichloromethane. 5 ml of 6 M hydrochloric acid was added thereto and the mixture was vigorously stirred for 1 hour. The resulting mixture was then centrifuged for 5 min at 5000 rpm. 1 ml of the supernatant was transferred to a new test tube and the tube was shaken at a rate of 60 times/min in a shaking water bath at 37° C. for 24 hours. 6 ml of 1 M sodium hydroxide was then added thereto and the mixture was shaken at a rate of 60 times/min in the shaking water bath at 37° C. for 24 hours. 50 μl of the resulting aqueous solution, 125 μl of 4% (w/v) sodium bicarbonate (pH 9.0) and 50 μl of 0.5% (w/v) trinitrobenzensulfonic acid solution were mixed together, kept at a room temperature for 2 hours, and then the concentration of the drug in the resulting mixture was measured with a microplate reader at a wavelength of 450 nm to determine the amount of the drug loaded into the microparticles. The drug loading amount and drug loading efficiency of the microparticles were calculated according to the following equations;

Drug loading amount (%)=(total amount of a drug encapsulated into microparticles/amount of microparticles)×100 <Equation 1>

Drug loading efficiency (%)=(total amount of a drug encapsulated into microparticles/total amount of drug used in the preparation)×100 <Equation 2>

The test results are shown in Table 1.

Table 1

TABLE 1

| | Drug loading amount | Drug loading efficiency |
|---|---|---|
| Sample | (%) | (%) |
| Example 1 | 7.53 | 87.31 |
| Example 2 | 10.93 | 96.93 |
| Example 3 | 11.21 | 97.93 |
| Example 4 | 11.91 | 99.31 |
| Example 5 | 9.85 | 98.32 |
| Example 6 | 7.56 | 86.53 |
| Example 7 | 10.33 | 99.39 |
| Example 8 | 10.53 | 98.19 |
| Comparative | 5.63 | 62.33 |
| Example 1 | | |

As shown in Table 1, the microparticles of the present invention have higher drug loading amount and loading efficiency than those of Comparative Example.

These results suggest that because SAIB which encapsulated the protein drug has a high viscosity, it blocks the outflow of the drug into the external phase when the microparticles are exposed to the external aqueous continuous phase.

Furthermore, increasing the amount of SAIB (Example 3) and adding the viscous water-soluble polymer such as starch, hyaluronic acid to internal aqueous phase (Examples 4, 5, 7 and 8) improved the loading amount and loading efficiency of the drug.

### TEST EXAMPLE 2

### In Vitro Drug Release Test

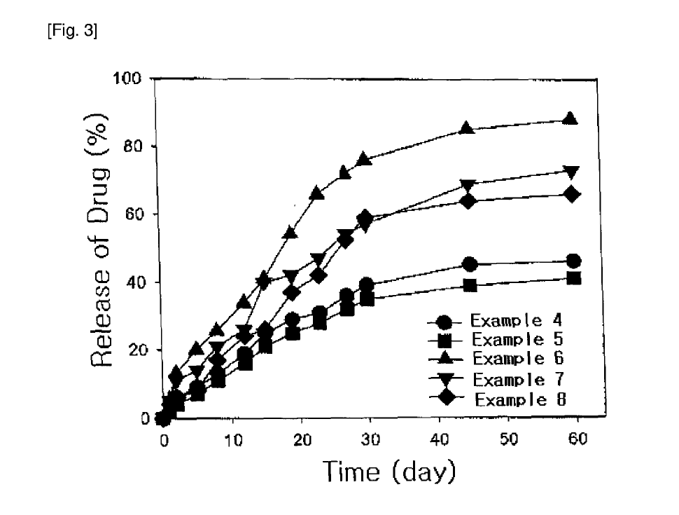

In order to examine the continuous controlled drug release characteristics of the microparticles prepared in Examples and Comparative Example, drug release tests were carried out as follows: 40 ng of microparticles was put in a test tube containing 10 ml of phosphate buffer (pH 7.4, 0.01% sodium azide, 0.02% Tween 80) capped, and shaken at a rate of 60 times/min in a shaking water bath maintained at 37° C. to allow the release of the drug for a period of 60 days or more. 5 ml of the buffer was taken hourly and the remaining buffer was supplemented with fresh phosphate buffer. The concentration of the drug released into the buffer was measured with a micro BCA reagent kit and the cumulative amount of drug released at each hour was calculated. The drug release rates thus obtained are shown in FIGS. 2 and 3.

As can be seen from FIGS. 2 and 3, the microparticles of the inventive Examples release the drug continuously without initial burst release of the drug, while the microparticles of the Comparative Example excessively released the drug at the early stage. FIG. 2, in particular, shows that for Comparative Example, the drug release was completed in 11 days after the initial burst release, while the inventive particles released the drug continuously for 30 days.

FIG. 3 shows the addition of polyethylene glycol and CAPTISOL™ to the internal aqueous phase improved the continuous drug release characteristics.

### TEST EXAMPLE 3

### In Vitro Drug Activity Test

40 ng of each of the microparticles prepared in Examples and Comparative Example was put in a test tube containing 10 ml of phosphate buffer (pH 7.4, 0.01% sodium azide, 0.02% Tween 80). The test tube was shaken at a rate of 60 times/min in a shaking water bath at 37° C. and fresh phosphate buffer was filled in the test tube one day prior to predetermined times. After collecting all amount of the drug releasing liquid at predetermined times, the remaining buffer was supplemented with a fresh phosphate buffer. The concentration of the drug releasing liquid was measured according to the same method as in Test Example 2. 150 μl of the drug releasing liquid was mixed with 100 μl of a cell suspension (66 mM of phosphate buffer, pH 6.2) with Micrococcus lysodeikticus (ATCC 4698) at a concentration of 0.5 mg/ml. An absorbance (measured at 450 nm) of the mixture was monitored at 15 second intervals every 4 minutes and the absorbance of a mixture versus the time is illustrated in a graph to calculate a relative activity (%) according to the following equations. The test results are given in Table 2.

Relative drug activity (%)=(early linear gradient for a drug released from the microparticles)/(early linear gradient for a drug)×100 <Equation 3>

Table 2

TABLE 2

| Sample | 1 day | 2 days | 4 days | 7 days | 14 days | 21 days | 28 days |
|---|---|---|---|---|---|---|---|
| Exam- | 99.77 | 99.35 | 98.03 | 97.93 | 97.62 | 97.16 | 96.25 |
| ple 1 | | | | | | | |
| Exam- | 99.93 | 99.56 | 98.12 | 98.02 | 97.93 | 97.21 | 96.32 |
| ple 2 | | | | | | | |
| Exam- | 99.97 | 99.32 | 98.52 | 98.33 | 97.69 | 97.49 | 96.11 |
| ple 3 | | | | | | | |
| Com- | 99.93 | 97.32 | 96.12 | 94.55 | 87.21 | — | — |
| para- | | | | | | | |
| tive | | | | | | | |
| Exam- | | | | | | | |
| ple 1 | | | | | | | |

As can be seen from Table 2, the microparticles prepared according to the inventive Examples show drug activity over a longer period of time than that of comparative Example.

While the embodiments of the subject invention have been described and illustrated, it is obvious that various changes and modifications can be made therein without departing from the spirit of the present invention which should be limited only by the scope of the appended claims.

1. A method for preparing a sustained-release microparticle comprising:
a) dissolving a protein drug in an aqueous solution to obtain a water phase;
b) dissolving sucrose acetate isobutyrate (SAIB) and a biodegradable polymer in an organic solvent to obtain an oil phase;
c) adding the water phase obtained in step a) to the oil phase obtained in step b) to form a primary emulsion; and
d) adding the primary emulsion to an external aqueous continuous phase to form a secondary emulsion and recovering the solid product formed in the secondary emulsion.

2. The method of claim 1, which further comprises adding an additive selected from the group consisting of a release-controlling agent, a stabilizer and a mixture thereof to the aqueous solution in step a).

3. The method of claim 2, wherein the release-controlling agent is selected from the group consisting of polyethylene glycol, polyoxyethylene sorbitan fatty acid ester, glyceryl monooleate, sorbitan fatty acid ester, hyaluronic acid, chondroitin sulfate, polyvinyl alcohol, starch, bovine serum albumin, chitosan, alginic acid, pectin, curdlan, gelatin, dextran, levan, glucan, polyhistidine, polylysine, poloxamer, glyceryl palmitostearate, benzylbenzoate, ethyloleate, soybean oil, cotton seed oil, sesame oil, peanut oil, canola oil, corn oil, coconut oil, rapeseed oil, theobroma oil, glycerin, mannitol, and a mixture thereof.

4. The method of claim 2, wherein the release-controlling agent is polyethylene glycol.

5. The method of claim 2, wherein the amount of the release-controlling agent ranges from 0.01 to 10 parts by weight based on 1 part by weight of the protein drug.

6. The method of claim 2, wherein the stabilizer is selected from the group consisting of a viscous water-soluble polymer, a cyclodextrin derivative and a mixture thereof.

7. The method of claim 6, wherein the viscous water-soluble polymer is selected from the group consisting of starch, cellulose, hemicellulose, pectin, lignin, chitosan, xanthan gum, alginic acid, pullulan, curdlan, gelatin, dextran, levan, hyaluronic acid, glucan, collagen, a salt thereof and a mixture thereof.

8. The method of claim 6, wherein the viscous water-soluble polymer is added to the aqueous solution to a concentration ranging from 0.1 to 10% (w/v).

9. The method of claim 6, wherein the cyclodextrin derivative is selected from the group consisting of 3-mono-o-methyl-cyclodextrin, 2,6-di-o-methyl-cyclodextrin, 2,3,6-tri-o-methyl-cyclodextrin, 2-hydroxyethyl-cyclodextrin, 2-hydroxypropyl-cyclodextrin, 3-hydroxypropyl-cyclodextrin, 6-o-glucosyl-cyclodextrin, 6-o-maltosyl-cyclodextrin, 6-o-dimaltosyl-cyclodextrin, 2,6-di-o-ethyl-cyclodextrin, 2,3,6-tri-o-ethyl-cyclodextrin, 2,3-di-o-hexanoyl-cyclodextrin, 2,3,6-tri-o-acetyl-cyclodextrin, 2,3,6-tri-o-propanoyl-cyclodextrin, 2,3,6-tri-o-butanoyl-cyclodextrin, 2,3,6-tri-o-hexanoyl-cyclodextrin, 6-o-carboxymethyl-cyclodextrin, sulfated cyclodextrin, sulfobutyl-cyclodextrin, a derivative thereof and a mixture thereof.

10. The method of claim 6, wherein the amount of the cyclodextrin derivative ranges from 0.1 to 20 parts by weight based on 1 part by weight of the protein drug.

11. The method of claim 1, wherein the protein drug is selected from the group consisting of lysozyme, human growth hormone, insulin, bovine growth hormone, porcine growth hormone, growth hormone releasing peptide, B-cell factor, T-cell factor, granulocyte-colony stimulating factor, granulocyte macrophage-colony stimulating factor, macrophage-colony stimulating factor, erythropoietin, bone morphogenic protein, interferon, atriopeptin-III monoclonal antibody, macrophage activating factor, interleukin, tumor degenerating factor, insulin-like growth factor, epidermal growth factor, tissue plasminogen activator, urokinase, protein A, allergy inhibiting factor, cell necrosis glycoprotein, immunotoxin, lympotoxin, tumor necrosis factor, tumor inhibitory factor, transforming growth factor, alpha-1 antitrypsin, albumin and its fragment polypeptide, apolipoprotein-E, factor VII, factor VIII, factor IX, pancreatic polypeptide, protein C, C-reactive protein, renin inhibitor, collagenase inhibitor, superoxide dismutase, platelet derived growth factor, osteogenic growth factor, osteogenesis stimulating protein, calcitonin, atriopeptin, cartilage inducing factor, connective tissue activating factor, follicle-stimulating hormone, luteinizing hormone, luteinizing hormone-releasing hormone, neurotrophic factor, parathyroid hormone, secretin, somatomedin, adrenocorticotropic hormone, glucagon, cholecystokinin, gastrin-releasing peptide, corticotropin releasing factor, thyroid stimulating hormone, monoclonal or polyclonal antibodies, virus derived vaccine antigen, leuprorelin acetate, goserelin acetate, nafarelein acetate, buserelin acetate, gonadorelin, Humira(adalimunab), Remicade(infliximab), octreotide acetate, a salt thereof and a mixture thereof.

12. The method of claim 1, wherein the biodegradable polymer is selected from the group consisting of the poly(acryloyl hydroxyethyl) starch, polybutylene terephthalate-polyethylene glycol copolymer, chitosan and derivatives thereof, polyorthoester-polyethylene glycol copolymer, polyethylene glycol terephthalate-polybutylene terephthalate copolymer, poly sebacic anhydride, pullulan and derivatives thereof, starch and derivatives thereof, cellulose acetate and derivatives thereof, polyanhydride, polylactic acid, polyglycolic acid, polylactic acid-polyglycolic acid copolymer, polycaprolactone, polycarbonate, polybutadiene, polyesters, polyhydroxybutyric acid, polymethyl methacrylate, polymethacrylic acid ester, polyorthoester, polyvinyl acetate, polyvinyl alcohol, polyvinyl butyral, polyvinyl formal, hyaluronic acid, lecithin, starch, protein and a mixture thereof.

13. The method of claim 1, wherein the biodegradable polymer has a weight-average molecular weight ranging from 2,000 to 100,000 daltons.

14. The method of claim 1, wherein the concentration of the biodegradable polymer in the organic solvent ranges from 5 to 60% (w/v).

15. The method of claim 1, wherein the weight ratio of the biodegradable polymer and SAIB used in step b) is in the range of 1:0.1 to 1:5.

16. The method of claim 1, wherein the organic solvent used in step b) is selected from the group consisting of dichloromethane, ethylacetate, dimethylsulfoxide, dimethylformamide, chloroform, alcohol, acetone and a mixture thereof.

17. The method of claim 1, wherein the volume ratio of the water phase and the oil phase used in step c) is in the range of 1:3 to 1:30.

18. The method of claim 1, wherein the external aqueous continuous phase in step d) is an aqueous polyvinyl alcohol solution.

19. A sustained-release microparticle comprising a peptide drug, which is prepared by the method according to claim 1.
